(19) United States
(12) Patent Application Publication
(10) Pub. No.: US 2009/0023804 A1
(43) Pub. Date: Jan. 22, 2009

(54) ELECTROCHEMICAL METHODS FOR REDOX CONTROL TO PRESERVE, STABILIZE AND ACTIVATE COMPOUNDS

(76) Inventors: Steven Baugh, Broomfield, CO (US); Thomas Hnat, Santee, CA (US)

Correspondence Address: CATALYST LAW GROUP, APC, 9710 SCRANTON ROAD, SUITE S-170, SAN DIEGO, CA, 92121, US

(21) Appl. No.: 12/181,285

(22) Filed: Jul. 28, 2008

Related U.S. Application Data
(63) Continuation-in-part of application No. PCT/US2007/061242, filed on Jan. 29, 2007.
(60) Provisional application No. 60/762,710, filed on Jan. 27, 2006.

(51) Int. Cl. A61K 31/353; A61P 31/12; A61P 31/18; C25B 9/00; B01J 19/08; A61N 1/30

(52) U.S. Cl. 514/456; 204/196.1; 204/248; 604/20

(57) ABSTRACT

To maximize and maintain the antioxidant or pro-oxidant state for foods, beverages, personal care products, cosmetics, nutritional supplements, reagents, analytical standards, medical device formulations, pharmaceutical preparations or drags, the present invention discloses methods and devices to control redox equilibrium of such preparations throughout the processing steps and storage prior to, or at, the time of administration or use. The preparations (solid or liquid form) can then be stored in a redox-controlled container, package or applicator as described in the specification. Foods, beverages, personal care products, cosmetics, nutritional supplements, reagents, analytical standards, medical device formulations, pharmaceutical preparations and drugs stored in reactive oxidation states can be activated and stabilized by electrical voltages applied with a small battery and electrodes designed into the applicator, container or package.

### RELATED APPLICATIONS

This application is a continuation of International Application Serial No. PCT/US2007/061242, filed Jan. 29, 2007, which claims the benefit of priority under 35 USC §119(e) of U.S. Provisional Application No. 60/762,710, filed Jan. 27, 2006.

### FIELD OF INVENTION

The invention of the present application relates to the field of physical and analytical chemistry, in particular to medicinal chemistry including electrochemistry and electrodynamic therapy, as used for achieving a specific redox equilibrium of a given substrate through electrical manipulation of redox properties, as well as stabilization of the desired redox state of a variety of materials. The methods of the present invention could be used in the following industries: food, beverage, personal care products, cosmetics, nutritional, reagent, analytical standards, medicinal, biochemical, pharmaceutical, manufacturing and other areas of the relevant technical arts.

### BACKGROUND OF THE INVENTION

This invention relates to methods of preparing, preserving and stabilizing foods, beverages, personal care products, cosmetics, nutritional supplements, reagents, analytical standards, medical device formulations, pharmaceutical preparations and drugs, more specifically, to methods by which a desired state of oxidation or reduction can be maintained in these products during extraction, purification, formulation, manufacture, packaging, administration, and storage thereof. This invention further relates to devices useful for providing and maintaining such redox-specific preparations. Additionally this invention can be used to stabilize reactive compounds by preventing continued degradation in preparations, thereby allowing their use and extending their shelf life.

Plants, for example, have an almost limitless ability to synthesize aromatic substances, most of which are phenols or their oxygen-substituted derivatives. In many cases these substances serve as plant defense mechanisms against predation by microorganisms, insects and herbivores. Some, such as terpenoids, give plants their odors; others, quinines and tannins, are responsible for plant flavor; and some of the same herbs and spices used by humans to season food yield useful medicinal compounds. Antioxidant properties of herbs have uses for anti-inflammatory and cancer prevention therapies, whereas pro-oxidant characteristics have found use for antibacterial, antiviral and cancer treatments.

Phenolic compounds are antioxidants (reducers) in that they contain redox-active molecules in reduced form. They can be subject to oxidation, the loss of an electron, forming free radicals and ultimately quinones. Thus, during their own oxidation they reduce oxidative challenges and protect biological substrates. Phenolic molecules behave as antioxidants in the reduced form and often as pro-oxidants in the oxidized phenolic radical and quinone forms. Many polyphenols compounds can repeatedly cycle non-destructively through the phenol, radical and quinone forms in a process called redox cycling. Many therapies subject photo-active compounds to light exposure, creating more efficacious oxidized forms in a process called photodynamic therapy.

Antibiotic, antiviral and anticancer products are all redox-active compounds, including free radical species (pro-oxidants) initiated during administration. Many antibiotic, antiviral and anticancer treatments involve the oxidation of the parent molecule and the production of other downstream reactive oxygen species. Vitamin C has been shown to be a pro-oxidant under elevated temperature conditions such as fever.

Many products including foods, beverages, personal care products, cosmetics, nutritional supplements, medical device formulations, pharmaceutical preparations and drugs contain polyphenolic structures within some of their ingredients that can oxidize to destabilize or degrade the compounds, formulation or product. Some phenolic compounds can be active pro-oxidants (oxidizers) or anti-oxidants (reducers), depending on manufacturing, storage conditions and use.

These and other compounds are subject to various manipulations involving chemical techniques (Vardosanidze et al., United State application Ser. No. 10/500,301). Such chemical manipulations can involve the addition of charged molecules, such as amino acids and their derivatives, in an effort to stabilize the redox properties of a given composition. Such a technique, however, relies on chemical manipulation of a composition to alter its redox properties. This results in the distinct disadvantage of having to go through necessary purification steps to rid the composition of the charged molecules needed to perform the chemical manipulation. There exists a need in the field to perform such manipulations in a way such that the composition is left unaffected from the addition of other chemicals.

There is a widely held belief in the notion that reduced forms of compounds are more efficacious than the oxidized form. One example is in the field of viral therapy, namely treatment of Human Deficiency Virus 1 (HIV-1) (Hamza and Zhan, 2006). Observers have noted that there would be great benefit in developing a potent inhibitor blocking the binding the glycoprotein CD4 (from the cell) with glycoprotein up 120 (from HIV-1), as this binding marks the initial phase of HIV-1 entry into cells. These observers have tested and measured, exclusively, the reduced form of epigallocatechin gallate ((−)EGCG) as a potential inhibitor of this CD4-gp120 binding. This compound, as well as additional anti-HIV therapeutics, is being developed with a focus on the reduced formulation of the drug being the most effective. However, there has never been a devotion to studying the oxidized forms of these compounds. Furthermore, a device does not exist in the art which would enable researchers to formulate either reduced or oxidized compounds, within specific parameters, in order to carry out such experiments. There exists a need in the field to have a device of the present invention in order to measure and develop precise formulations of redox-adjusted compounds for advancements in antibiotic, antiviral and anticancer therapeutics.

These and other compounds are subject to electrochemical manipulation utilizing a variety of specific techniques including, but not limited to, cyclic voltammetry, linear sweep voltammetry, bulk electrolysis, normal and differential pulse voltammetry, normal/differential pulse polarography, stripping voltammetry, chronopotentiometry and other like techniques as applied by those skilled in the art. In cyclic voltammetry, the molecule must undergo reversible oxidation and reduction in a process called redox cycling. During the analysis, the compound is repeatedly oxidized and reduced electrochemically, creating all forms: reduced phenol, semiquinone radical, and quinone. Industry needs predictable redox control and verification possible with electrochemical equipment to investigate the various controlled forms of redox-active medications.

There is a present need for the application of methods for stabilizing the redox state during the preparation of a given material so that a desired redox state can be maintained during manufacture, storage, consumption, administration or use. To this end, the present invention describes methods and devices useful for providing foods, beverages, personal care products, cosmetics, nutritional supplements, reagents, analytical standards, medical device formulations, pharmaceutical preparations and drugs with a desired redox state, either reduced or oxidized.

### BRIEF SUMMARY OF THE INVENTION

In one aspect, the present invention provides a container or package for maintaining an oxidizable or reducible compound in a desired redox state, the container comprising an anode and a cathode in electrically conductive contact with said compound, the anode and cathode being in electrical contact with a source of electromotive force, said source supplying sufficient electromotive force to maintain the compound in a desired redox state when in contact with the anode or cathode. Preferably, the container provides the source of electromotive force as a battery, piezoelectric or other voltage source. More preferably, the anode within the container has a greater surface area than the cathode. Alternatively, the cathode within the container has a greater surface area than the anode. Still more preferably, the container includes a redox electron sink.

In one aspect, the present invention provides a manufacturing scale electrochemical cell capable of large scale redox regulation of chemicals. This equipment can be used to manipulate the redox state of a large batch of chemical, and then the redox state can be maintained by the embodiment described above. Producing an oxidizable or reducible compound in a desired redox state, the vessel or component of a flow process, comprising an anode and a cathode in electrically conductive contact with said compound, the anode and cathode being in electrical contact with a source of electromotive force, said source supplying sufficient electromotive force to produce the compound in a desired redox state when in contact with the anode or cathode. Preferably, the container provides the source of electromotive force as an industrial or other voltage source. More preferably, the anode within the container has a greater surface area than the cathode. Alternatively, the cathode within the container has a greater surface area than the anode. Still more preferably, the container includes a redox electron sink. There are many examples of electrochemical processes achieving the same chemical reactions and syntheses as various chemical redox manipulations.

In another embodiment the electrochemical equipment can be used to stabilize a chemical reaction process. During oxidation (pH, photochemical, metal catalyzed, enzymatic, etc.) electrons are liberated during the oxidation. If the electrons are not consumed they are free to react, with water and dissolved oxygen to form downstream reactive species such as hydrogen peroxide, superoxide radical and hydroxyl radical. These downstream reaction products can continue oxidation processes and affect the storage and shelf life of chemically modified products. Electrochemistry equipment can be used in conjunction with chemical oxidations to essentially measure and then maintain an existing electron “pressure” in the solution. As additional electrons are liberated they can be absorbed by the electrochemical circuit, preventing destructive downstream reactive species either real time or as a final polishing step. This was demonstrated by the increased stability of the 320 nm quinone absorbance in the electrochemically modified solutions (FIG. 9).

In another aspect, the present invention provides a method of utilizing the concept of control of gene expression, as controlled by redox-active compounds. Specifically, the expression of genes has been shown to be effectively regulated by redox-active compounds (Kauffmann et al., 2002.) As such, the present invention provides a means of identifying, formulating, and administering redox-active compounds in order to control the expression of particular genes and other redox-regulated biological processes associated with disease (see Halliwell, Free Radicals in Biology and Medicine, Oxford University Press. USA; 3rd edition (1999) which is herein incorporated by reference).

Analytical techniques involving electrodes have been utilized in order to electrochemically reduce or oxidize, for example, antibiotics (Oliveira et al, 1999; Ozkan et al., 2002) This supports the notion that such electrochemical modification may not only be used for therapeutic and manufacturing purposes, but may also be done for analytical purposes. For example, use of such electrochemical modification of antibiotics may enable one to understand the molecular mechanisms behind the action of certain antitumor therapeutics. Comparisons may then be made between several antitumor agents to gain a deeper understanding of the mechanisms and effects of such agents as both controlled pro-oxidants and anti-oxidants.

In yet another aspect, the present invention provides a method of compound preparation for the modulation of tumor progression, it has been implicated that reactive oxygen species play important roles in modulating tumor progression (Savaraj et al., 2005). While Savaraj et al. imply that antioxidant modulation of antitumor progression may be contributed by the down-regulation of expression of metalloproteins, it is appreciated that the concept of redox regulation may be used in order to modulate tumor progression. The present invention provides a means of such alteration, thereby leading to novel approaches to producing effective antitumor therapeutics.

In another aspect, the present invention provides an applicator for preparing and/or administering an oxidizable or reducible compound in a desired redox state comprising: (1) dissolving or suspending the compound in an electrically conductive solution; (2) contacting the solution containing the compound with an anode and a cathode; and (3) supplying a measured electromotive force to the anode and cathode, said force being sufficient to oxidize or reduce the compound to a preferred or desired state. In one preferred embodiment, such preferred or desired state of the compound is prepared and administered. Preferably, the applicator is one selected from the group consisting of: (1) a skin patch; (2) an eyedropper; and (3) a syringe or other forms of electrodes.

The present invention will provide a new way to produce, stabilize, maintain and activate drug preparations that are subject to oxidation and reduction reactions. The potential outcomes of the new technology are several-fold:
1) The present invention can potentially extend the shelf life of drugs and guarantee they are in the redox state in which they were produced.
2) The present invention will be able to produce compounds in a known oxidation state which will allow researchers to demonstrate which formulations might be more effective in their pro-oxidant state. This concept represents a departure from the traditional view that antioxidant compounds should be used in their reduced form. Currently oxidation state and reduction potential of the medicine is not monitored directly and often not part of the drug discovery process.
3) The present invention does not require the addition of secondary oxidizing or reducing substances to achieve the desired redox state as in traditional chemical redox manipulations. The redox state can be measured by an external voltammeter, color changes, spin-trap dyes, UV-visable spectroscopy, fluorescence spectroscopy, ESR/EPR spectrometry, or mass spectrometry, among other techniques.

### BRIEF DESCRIPTION OF THE DRAWINGS

FIG. 1 exemplifies the reversible two-electron oxidation of quercetin, with the loss of two electrons and two protons.

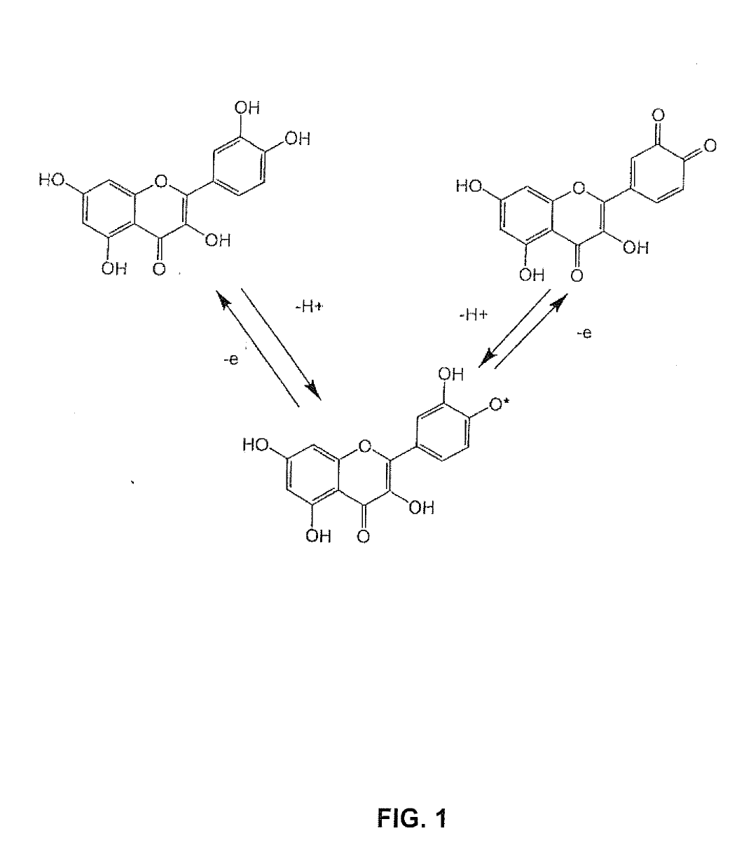

FIG. 2 shows the structures of exemplary phenolic compounds that are active ingredients of herbal preparations.

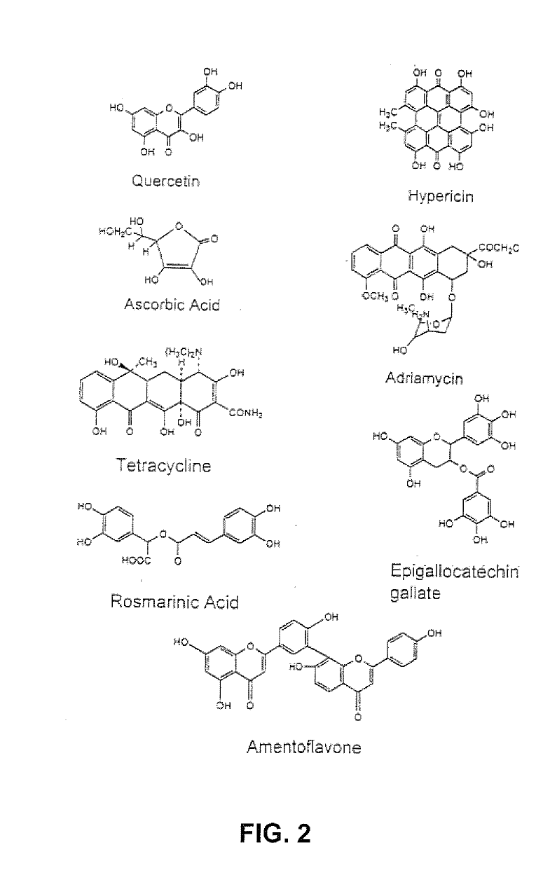

FIG. 3 shows examples of the phenolic compounds that can make square planar complexes consisting of two catechol groups, and coordinate transition metal ions, particularly divalent ions.

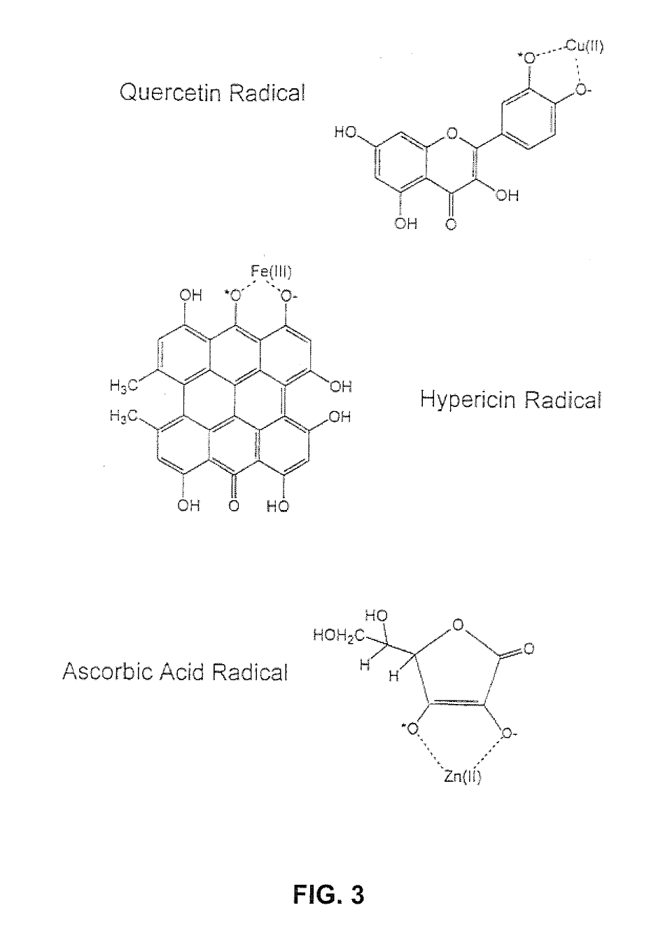

FIG. 4 shows an example of a bag in which a sample is introduced unidirectionally into a mixing chamber and then filtered while maintaining a desired or preferred redox state.

FIG. 5 shows cyclic voltammograms of hypericin. Ic= cathodic current; Ia= anodic current; the solvent used is DMF; the supporting electrolyte is n-Bu₄NPFf₆; the temperature is 298 K; the working electrode is a platinum disk; the counter electrode is a platinum wire; the scan rate is 0.25 V/s.

FIGS. 6A-6C depict bottles for storing a liquid capable of supporting electrical current and oxidative reactions. The voltage can be applied at the electrodes constantly or as determined by a switch built into the bottle. In an another embodiment, the container can be a flame-sealed vial under which a protective gas can work in conjunction with a battery in the bottom of the vial and electrodes to stabilize the solution.

FIGS. 7A and 7B show an eyedropper and a syringe with electrodes to activate a subsample of the liquid medicament, avoiding potential polymerization problems during storage.

FIG. 8 is a UV-visible spectrum analysis of EGCG reduced and oxidized absorption peaks after electrochemical treatment in a battery-in-a-bottle apparatus.

FIG. 9 is a UV-visible spectrum analysis of EGCG reduced and oxidized absorption peaks after electrochemical treatment and storage for one day and seven days.

FIG. 10 shows the effect of reduced and oxidized EGCG on the survival of herpes simplex 1 and 2.

FIG. 11 shows the direct infusion mass spectral data of EGCG samples oxidized by classical wet chemical means. The compound with mass 911 has been identified as the EGCG dimer quinone, a predictable oxidation product of EGCG. These masses, and mass 911 in particular, are also present in the electrochemically oxidized sample analyzed by direct infusion mass spectrometry, indicating the formation of analogous larger molecules by phenolic coupling and free radical addition mechanisms.

FIG. 12 depicts a diagram of electrons lost during oxidation.

### DETAILED DESCRIPTION OF THE INVENTION

Unless defined otherwise, all technical and scientific terms used herein have the same meaning as is commonly understood by one of skill in the art to which this invention belongs.

The following definitions are provided in an effort to remove any ambiguities relative to the interpretation of the present application.

The term “antioxidant” as used herein refers to any substance that inhibits the effects of oxidation, thereby leaving the other materials, compounds or preparations in a mixture in a reduced state. Likewise, the term “pro-oxidant” refers to any substance that promotes the effects of oxidation, thereby leaving the other materials, compounds or preparations in a mixture in an oxidized state.

The term “oxidizing agent” as used herein refers to any substance, process, light exposure, applied voltage, magnetic fields, or chemical species that causes another material to be oxidized, thereby leaving the oxidizing agent in a reduced state by accepting the electrons removed through oxidation. Likewise, the term “reducing agent” refers to any substance or chemical species that causes another material to be reduced, thereby leaving the reducing agent in an oxidized state by losing the electrons donated during the reduction. Examples of oxidizing agents or conditions that promote oxidation are transition metal ions, oxygen, ozone, light, applied voltages, applied magnetic fields, and elevated pH.

The term “redox potential” as used herein is a measure of the tendency of a solution to remove or add (oxidize or reduce, respectively) electrons. The redox potential may also be described as the electron pressure that the electrochemical cell exerts. The redox potential (Eh) is measured electrochemically and expressed in units of electrical potential difference (e.g., volts): the more positive the number of volts, the higher the relative concentration of oxidant to reductant in solution, and vice versa.

The term “redox state” as used herein is the condition of the molecule that is manipulated or controlled electrochemically. The electrochemical modification of the redox state to the most effective oxidation state (i.e., phenol, radical, or quinone) is one aspect of the present invention.

The objective of the invention is to provide foods, beverages, personal care products, cosmetics, nutritional supplements, reagents, analytical standards, medical device formulations, pharmaceutical preparations and drugs in a desired or controlled redox state which, as a result, will have improved activity, reproducibility, predictability, stability and shelf life. The present invention provides methods and devices for controlling redox equilibrium during formulation, manufacture, packaging, administration and use of such products and preparations. Some active polyphenolic compounds contained within the food, beverage, personal care product, cosmetic, nutritional supplement, medical device formulation, pharmaceutical preparation or drug can function as either a pro-oxidants or antioxidants, depending upon the redox state therein. A pro-oxidant product prepared according to the method herein will have an oxidizing affect. An antioxidant product prepared according to the method herein will have an antioxidant effect. Many polyphenolic compounds can cycle between antioxidant and pro-oxidant redox states, Redox cycling polyphenolic compounds in foods, beverages, personal care products, cosmetics, nutritional supplements, reagents, analytical standards, medical device formulations, pharmaceutical preparations and drugs that have been oxidized by natural or other means can be reduced using an electromotive force to change the specific redox state of the polyphenolie substance to the reduced form as necessary.

Many biological and pharmaceutical molecules participate in oxidation-reduction (redox) reactions. Solutions of these molecules in polar, particularly aqueous, solvents have various degrees of oxidation based on a variety of variables that are difficult to control such as light, heat, trace levels of transition metal contamination, and time of storage. Strict control of these variables is necessary to control the extent of oxidation of analytical standards, medical preparations, intravenous and other solutions.

In one aspect of the present invention, electrical stimulation of compounds results in the ability to manipulate the redox state or redox state equilibrium, and therefore the properties of such compounds. Such manipulation allows the invention to transition between oxidized and reduced states of the compound of interest. In particular, the compound is a substance involved in the food, beverage, personal care products, cosmetics, nutritional, reagent, analytical standards, medicinal, biochemical, pharmaceutical, manufacturing and other areas of the relevant technical arts.

Most polyphenolie compounds contained within foods, beverages, personal care products, cosmetics, nutritional supplements, reagents, analytical standards, medical device formulations, pharmaceutical preparations and drugs currently in use are prepared without any mechanism(s) to maintain or monitor the redox state of the active ingredients at each stage of the preparation. Chemical ingredients are normally added to the formulation to prevent oxidation and maintain a reduced redox state over the shelf life of the product or preparation. Therefore, the manufacturing conditions or composition generally determines the redox state of the products. In order to provide a product in a desired redox state, the redox potential of a given preparation is measured. The redox state of the product may then be adjusted throughout the entire preparation steps including storage, time of administration and use. The redox state of a food, beverage, personal care product, cosmetic, nutritional supplement, reagent, analytical standard, medical device formulation, pharmaceutical preparation or drug at a particular stage of preparation can be measured by stepping or cyclic voltammetry in conjunction with UV-visible spectroscopy, fluorescence spectroscopy, or electron spin resonance (ESR) as described herein to determine and then monitor a desired redox state.

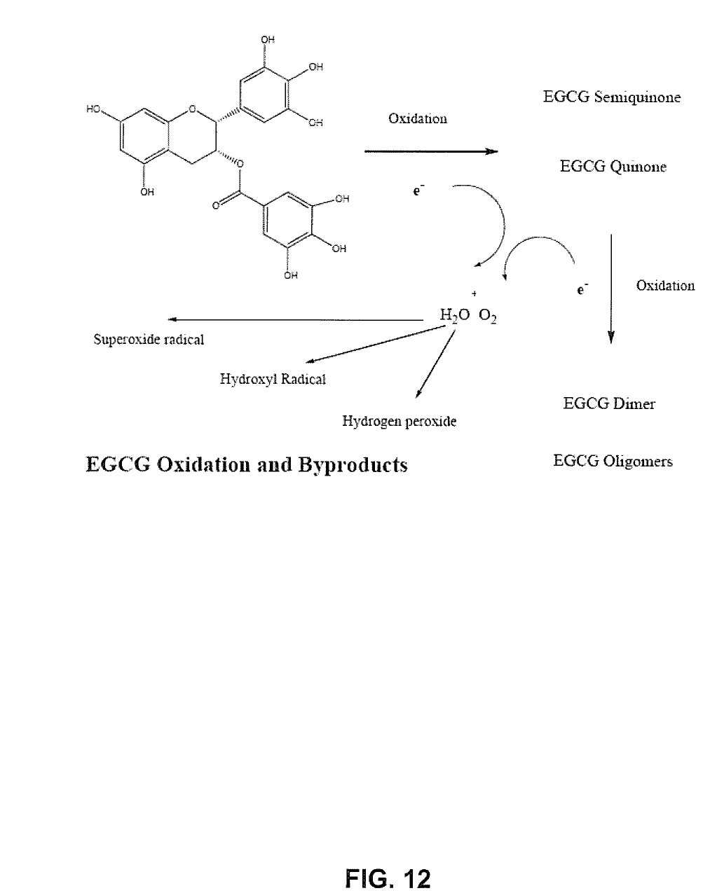

The invention further provides containers, packaging and applicators that are capable of preparing, maintaining, and administrating reducing or oxidizing compounds in a desired redox state by electrochemical means. Additionally this electrochemical technology can be used to stabilize redox-active chemicals in solution, such as quantitative and qualitative solutions of reference materials. Electrons are lost during oxidation, these electrons either react directly with the oxidizer or they are lost into solution, where they can react with dissolved oxygen to form superoxide radical or they react with water and oxygen to form hydroyl radicals and hydrogen peroxide. The invention controls the free electrons during the manufacturing, storage and application of the activated materials to ensure that destructive downstream reaction products such as superoxide radical, hydroxyl radical and hydrogen peroxide do not interfere with the activation and/or control of the redox application, be it photochemical, classic wet chemistry, applied electromagnetic fields or other oxidation processes (FIG. 12).

One aspect of the present invention involves containers and applicators that are capable of preparing and maintaining reducible or oxidizable compounds in a desired or preferred redox state by electrochemical means. One such example is a battery included in a container or applicator in such a way that an anode and a cathode in electrically conductive contact with the compound and with a source of electromotive force generates sufficient electromotive force to maintain said compound in the desired or preferred redox state. There is a relationship between the surface area of the electrodes relative to the volume of solution within the container. If the distance between the anode and cathode is small and the surface area is large, then only a small amount of fluid may be held between the two surfaces, resulting in a high surface area to volume ratio, such as the interface area of a ball and socket joint. If the distance between the anode and cathode is increased, the surface area remains the same while the volume of the fluid which may be contained between the two points increases, resulting in a diminished surface area to volume ratio. Most preferably, there should be a maximization of the surface area to volume ratio in order to maximize reactions taking place at the surface of the anode and cathode. Such a container or application is particularly useful where the desired redox state is difficult to maintain without including an undesirable compound in the formulation.

The container can be used for storage of solutions, as a step in a manufacturing process, or for activation of foods, beverages, personal care products, cosmetics, nutritional supplements, reagents, analytical standards, medical device formulations, pharmaceutical preparations and drags at the time of use. The containers can be screw-topped, flame-sealed or any other convenient container configuration. In addition, the redox control can be exerted during manufacture as a flow system by incorporating electrodes into the flow process. For longer processes and long-term storage, it may be necessary to introduce a semi-permeable membrane to allow the electrons to flow between the working electrode and the counter-electrode compartments while keeping the solutions at each electrode from mixing, as in the bulk electrochemical cell design currently in use.

An aspect of the present invention provides for a method of designing a compound or substance in a preferred or desired condition through the measurement and adjustment of the redox state of the compound or substance during the preparation, including storage and administration of the compound or substance. The redox potential indicates the level of oxidizing and reducing power of a compound or substance. Therefore, the desired redox state of a preparation is selected according to the preferred or desired therapeutic effects intended for use of the compound or substance as a medicinal product. Alternatively, a preparation itself may be manipulated through the methods described above in order to achieve a more stabilized form of medicinal product.

In order to maintain a desired redox state of any medicinal product during the preparation stage, the redox potential of a preparation is measured at each stage and adjustments to the redox state are performed as needed based on the preferred or desired redox state to be achieved. Such adjustments are accomplished by the methods described herein.

The container can also be a syringe wherein the solution is electrochemically optimized during the injection of the drug. Additionally the electrochemical redox control technology can be used in conjunction with reactive topical or internal medications, in a process called electro-dynamic therapy, by using miniature electrodes strategically placed at two ends of a targeted area to maintain a desired redox state. Examples include skin patch electrodes or localized electrodes for activation of medications at a specified site. Electrodynamic therapy itself or in combination with stabilized reactive preparations could be particularly useful for treating skin lesions or in combination with surgical treatment or removal of tumors while the body cavity is open. With the use of nanotechnology the potential exists to maintain a desired redox state at the cellular level. For example, applying the principle of changing or reversing the redox state, nano-electrodes with an electromotive force can apply electrodynamic therapy across aggregates of cells or tumors targeted for removal or destruction. Pro-oxidant or antioxidant compounds injected into the tumor can be continuously maintained in a specified redox state with nano-electrodes until the condition is reversed or eliminated.

Data suggest that antioxidant compounds can behave as pro-oxidants given the proper conditions and redox state. Antioxidant treatments are well understood and accepted, however there are also pro-oxidant antibiotic and chemotherapeutic agents currently in use. The fact that the same compound can have different efficacy based solely on the oxidation state illustrates the importance of redox state regulation for optimal efficacy. The body's immune response can be supplemented with reactive or protective species, pro-oxidant or antioxidant, to either protect or target problem areas internally or topically. Antioxidant medicinal preparations can be optimized and used alone or in combination with pro-oxidant preparations to quench after-effects of an oxidative treatment such as chemotherapy after a specified time period has elapsed (i.e., sequential administration).

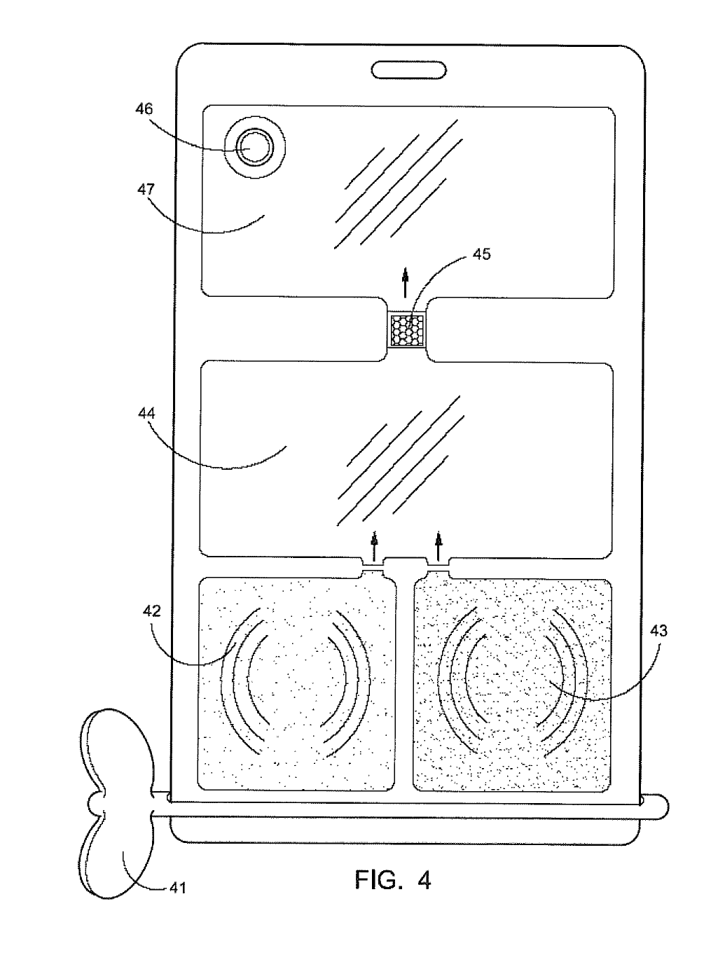

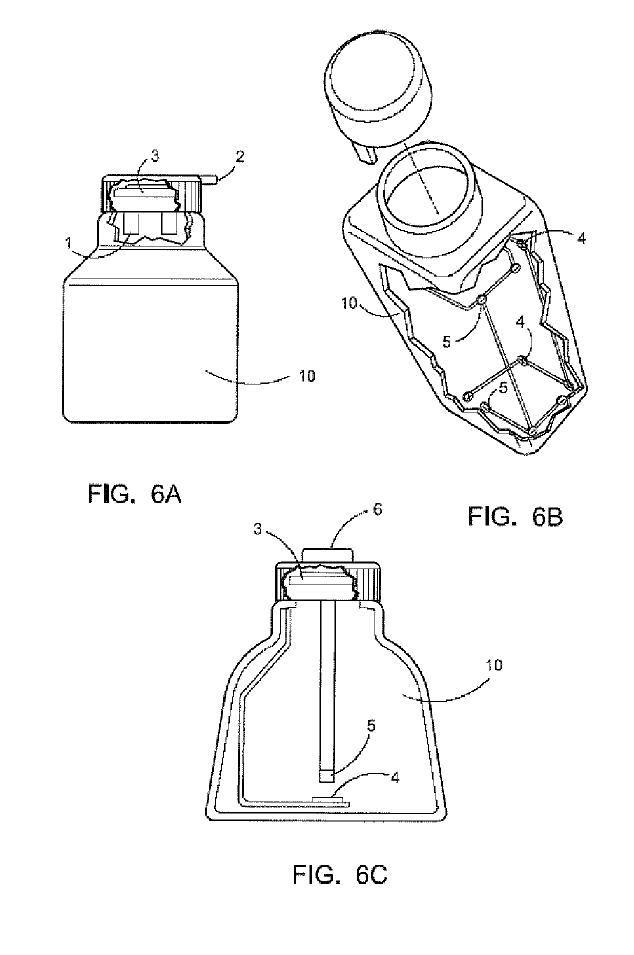

The invention also provides a container or other packaging for storing, preparing or administering preparations prior to or at the time of use in a desired redox state. Such a container or package (i.e., bottle or I.V. bag) includes a source of electromotive force such as a battery to maintain the food, beverage, personal care product, cosmetic, nutritional supplement, reagent, analytical standard, medical device formulation, pharmaceutical preparation or drug in a desired redox state. The sample in such a container is dissolved in a conductive salt solution, i.e., electrolyte, which can be buffered to maintain a desired pH, and in electrical contact with the battery. FIG. 4 depicts a bag with two compartmentalized reagents, reagent A42 and reagent B43, with a built-in filter 45 to remove particulates after mixing. There is a means 41 attached to the bag for the purpose of forcing fluids (e.g., rolling) or for preventing backflow. Reagents A and B are mixed in the mixing chamber 44, passed through a filtering device 45 and are then ready to be connected to a syringe or intravenous tube at 46. FIG. 6 illustrates other examples of such containers; each bottle as shown contains a battery 3 on the lid with or without a switch 6 which can be easily accessible; i.e., lift tab 2. Bottles as shown have a ‘tongue and groove’ locator for supplying electromotive force to the cathode 4 and anode 5.

FIG. 6B illustrates a bottle with electromotive force in contract with the entire inner surface so that the bottle can be used in any position and still be in contact with the solution. The voltage can be applied at the electrodes constantly or as determined by a switch 6 built into the bottle (FIG. 6C). The voltage to be applied for a given product or preparation is predetermined by cyclic voltammetry or other suitable method. For example, the voltage suitable for most foods, beverages, personal care products, cosmetics, nutritional supplements, reagents, analytical-standards, medical device formulations, pharmaceutical and drug preparations can range from 0 to 3 volts.

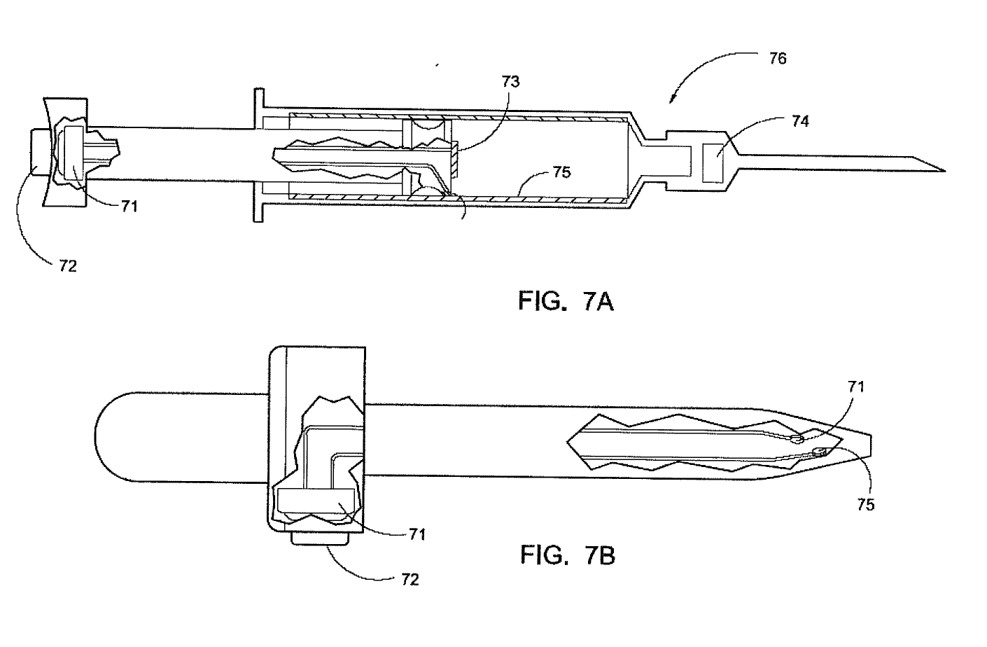

The container can also be constructed to serve as an adaptor for a syringe or eyedropper for application as illustrated in FIGS. 7A and B, respectively. In this case, the syringe needle can serve as an anode for oxidizing or reducing the sample at the time of administration. FIG. 7A shows a syringe with a battery 71, a switch 72, a plunger serving as the cathode 73, a needle as the anode 75, and a filter 74 to eliminate any undesirable precipitates. The syringe body 76 can be either metal or plastic, FIG. 7B is an eyedropper constructed similarly showing the cathode 75, the anode 71, a battery 71 and a switch 72. An added advantage of such a container (or an applicator) is its ability to cycle, i.e., cyclic reduction followed by oxidation, which will minimize any polymerization and additional reactions during storage.

Conductive polar solutions of redox-active phenolic compounds can be prepared for topical activation and treatment. There are currently medications and treatments that involve the use of polar-conductive gels and lotions containing compounds stimulated with skin surface electrodes as used in iontophoresis. This strategy can be applied to a topical medical preparation using this invention. For example, a skin patch containing an oxidized form of a preparation can be continually oxidized at the time of application by applying electrical potential. This treatment can be useful for a variety of uses including, antiviral applications, skin cancers, and other topical applications requiring reactive, oxidized compounds. This is novel and different than electrochemotherapy in that, electro chemotherapy focuses on voltages and pulses that modify the cell wall and increase uptake of the chemotherapy drag. This application specifically targets the chemotherapy molecule and the modification of redox state for increased efficacy.

The Examples that follow illustrate preferred embodiments of the present, invention and are not limiting the specification and claims in any way.

### EXAMPLE I

### Analytical Techniques for Compound Measurement

The structure of most molecules to be investigated incorporates aromatic rings with multiple phenol sites for oxidation. One common example would be epigallocatechin gallate (EGCG), EGCG has two adjacent phenolic groups that can oxidize once to form the semiquinone, and a second time to form the quinone. These three oxidation states are the dominant forms under physiological conditions.

The phenolic material can be monitored using a UV absorbance of the solution with appropriate blank. Phenols with moderate conjugation typically have strong UV absorbance in the 260 to 280 nm region; EGCG has an absorbance peak at 272 nm. Direct comparison of standard solutions to formulation solutions with increasing degrees of oxidation will be indicated by a corresponding decrease in relative reduced phenol concentration.

The quinone is the second oxidation product. In the case of EGCG, adjacent phenolic groups yield adjacent quinones. These molecules typically exhibit strong fluorescence. The UV absorbance of the quinone also shifts up to between 280 nm and 350 nm with emission wavelengths exceeding 400 nm. EGCG quinone has an absorbance peak at 320 nm. The specific excitation and emission wavelengths could be quickly determined using forced oxidation experiments and a relationship between concentration and intensity developed to monitor concentrations in the formulation.

The semiquinone radical intermediate is more difficult to analyze directly. The most direct approaches are electron spin resonance (ESR) and electrochemistry, in ESR, a free radical is monitored directly by application of an external magnetic field that flips the spin from +½ to −½ (unpaired). The movement of the electron between states gives off light of characteristic wavelength. This is similar to nuclear magnetic resonance (NMR), the difference being NMR flips nuclear spins with an external field.

### EXAMPLE II

### Electrochemical Oxidation, Storage and Activation

To ensure that a medicinal preparation of the present invention is in a preferred or desired redox state at the time of manufacturing, storage and administration, the preparation can be initially manufactured electrochemically and then stored in a container (e.g., bottle or intravenous delivery bag) with a battery. This battery may be activated at the time of administration or, more preferably, before administration. Any medicinal preparations can thus be electrochemically oxidized or reduced at any stage prior to or at the time of administration.

In order to stabilize a compound, substance or preparation, a constant voltage is applied to the medicinal formulation, this formulation comprising either or all of the following; compound(s), substance(s) or preparation(s). Alternatively, an electrical potential can be applied at the time of dispensing from the bottle or applicator.

For topical applications, the medicinal formulations can be activated electrochemically at the time of the application using a commercially available skin surface electrode. One instance of this present example would be utilizing these topical skin electrodes in combination with, conductive carrier solutions or skin patches, which can utilize nine-volt battery technology currently used with corticosteroid treatment.

In order to maintain a preferred or desired redox state and also to prevent possible polymerization or radical addition reactions during storage, the formulations may be stored in a container in which an active ingredient is cycled electrochemically in the presence of transition metal ions (e.g., Cu²⁺) through oxidation-reduction states so that the preferred or desired redox state of the medicinal formulation can be achieved at the time of administration.

### EXAMPLE III

### Oxidation States as Measured Electrochemically

The multiple oxidation states of the molecule of interest can also be determined electrochemically. In the case of EGCG, the molecule can be cycled repeatedly through the various oxidation states. This electrochemical technique is called cyclic voltammetry. The first phase involves applying an excessive positive potential (oxidizing potential) and oxidizing all the species present to one form. Then the applied voltage is stepped down in increments while monitoring current. When the redox potential of a reaction is approached, molecules begin to be reduced, causing current through the circuit. After returning all molecules to the reduced form, the voltage is increased and the redox potentials of the reaction(s) are recorded.

Additionally, the open circuit potential of the solution versus a standard reference electrode can be measured. This method utilizes two electrodes, working and reference, to determine the electrochemical potential generated by the solution versus a reference electrode, such as Ag/AgCl. This method will give an indication of the overall degree of oxidation of the formulation and when combined with cyclic voltammetry can be used to determine the species present.

By knowing the electrochemical potentials that create the semiquinone and quinone species, an external electrical potential can be applied to drive the redox state of the formulation to a predetermined oxidation state. Continuous application of the electrical potential will maintain the formulation in the desired oxidation state. This electrochemical potential can be supplied from a battery, small circuit and electrodes designed into an existing container format.

This concept is analogous to an extended DC Potential Amperometry (DCPA) experiment. In a DCPA analysis a constant potential is applied to the electrochemical cell, and the resulting current is measured. As long as there are reactions, a current will be observed. The experiment is terminated when the current goes below a predetermined level at that potential and all species are converted to the form selected by the potential. Similarly, the present invention is designed to achieve zero current (all in one form) and maintain the potential.

This electrochemical control requires a supporting electrolyte. There are several buffer systems that have physiological pH ranges and support electrochemical reactions, including TRIS and HEPES buffers. Their conductivity can be increased by addition of 10 mM CaCl₂ if necessary.

### EXAMPLE IV

### Measurement of Redox Potential

In order to provide a medicinal preparation in a preferred or desired redox state, the redox potential of the preparation at a given stage must be measured first and then the redox state may be adjusted according to the desired condition. The redox potential must be monitored, preferably in real time, and more preferably by a device.

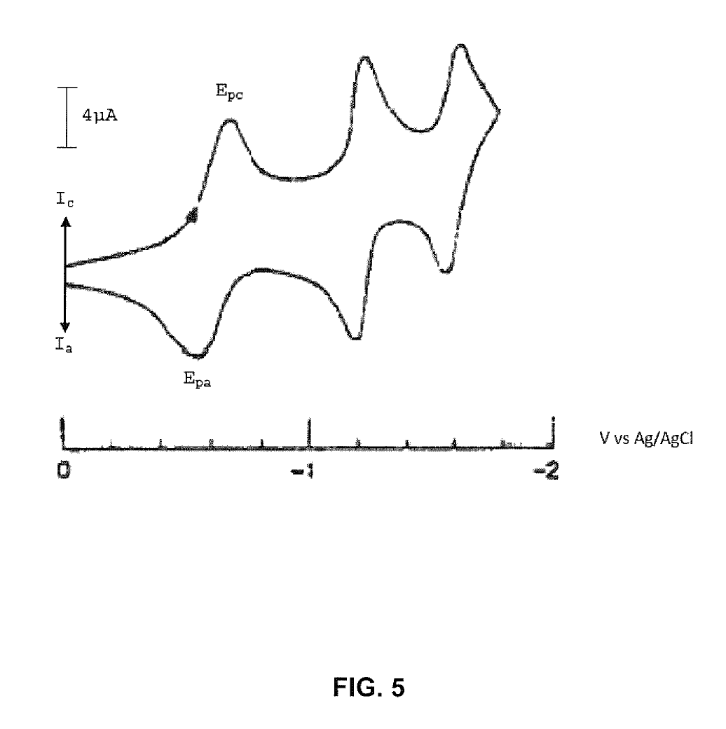

The redox potential of the solution is measured by open cell potential. Cyclic voltammetry modifies the sample as it is repeatedly cycled through multiple oxidation states and does not measure the current redox potential of the solution. Cyclic voltammetry can be used to identify the various oxidation states and redox potentials for future manipulation of redox state. Additionally, FIG. 5 in the present application provides a working example of this assay by depicting the cyclic voltammetry trace for hypericin. As shown, there are multiple peaks in the range of 0-2 volts, that are the result of the different redox states of hypericin. This is manipulated according to the preferred or desired redox state to be achieved.

If a detailed analysis of the oxidation status and molecular distribution of a medicinal preparation is needed, one may utilize other techniques such as electron spin resonance (ESR) spectrometry, UV-visible spectroscopy, fluorescence spectroscopy, mass spectrometry, gel permeation chromatography (GPC), or any other technology known in the art. Assays performed by each technology provide a different set of information; ESR can measure relative concentration of radicals in the sample, thereby providing an analytical technique in order to accomplish purification steps in the preparation; GPC and mass spectrometry measure the molecular weights of the compounds; UV-visible and fluorescence spectroscopy quantify the amounts of phenolic and quinone species present in the preparation.

Once the redox potential of the preparation is measured, the redox state may be adjusted to the preferred or desired condition at any stage as previously described. Table I provides examples of reduction potentials measured with the model compounds.

TABLE 1
Reduction Potentials for Selected Model Compounds

| Compound | Reduction Potential (mV)* |
|---|---|
| Quercitin | 120 |
| Caffeic Acid | 120 |
| Epigallocatechin gallate | 430 |

*= pH 7 Buffer solution versus Normal Hydrogen Electrode (NHE)

### EXAMPLE V

### Oxidation States Modulated and Measured Electrochemically

The multiple oxidation states of the molecule of interest can be determined electrochemically by cyclic voltammetry. The first phase involves applying an excessive positive potential (oxidizing potential) and oxidizing all the species present to one form. Then the applied voltage is stepped down in increments while monitoring current. When the mid-point redox potential of a reaction is approached, molecules begin to be reduced, causing current through the circuit. After returning all molecules to the reduced form, the voltage is increased and the redox potentials of the reaction(s) are recorded. This technique has been employed by others to determine redox states of EGCG (Kilmartin and Hsu, 2003).

Additionally, the open circuit potential of the solution versus a standard reference electrode can be measured. This method utilizes two electrodes, working and reference, to determine the electrochemical potential generated by the solution versus a reference electrode, such as Ag/AgCl. This method will give an indication of the overall degree of oxidation of the formulation, and when combined with cyclic voltammetry, can be used to determine the species present.

By knowing the electrochemical potentials that create the semiquinone and quinone species, an external electrical potential can be applied to drive the redox state of the formulation to a predetermined oxidation state. Continued application of the electrical potential will maintain the formulation in the desired oxidation state. This electrochemical potential can be supplied from a battery, small circuit, and electrodes designed into a container format (battery-in-a-bottle). The reduction potential of EGCG at pH 7 is 430 mV versus a normal hydrogen electrode.

An electrochemical cell was fabricated using a fine porosity glass frit in 1″ diameter glass tubing. The electrochemical cell houses the counter electrode and keeps the Ag/AgCl reference electrode and platinum working electrode separate from the counter electrode. This addition to the system allowed the preparation of bulk solution. The working electrode and reference electrode compartment was continuously stirred using a small stir bar and a magnetic stirrer to minimize gradient effects at the working electrode.

The EGCG solutions were oxidized using a positive potential to a visual end point. UV-visible spectral data were collected during these experiments, including the absorbance at 270 nm and 320 nm, corresponding to the phenol and quinone forms of EGCG, respectively. In addition, the open cell potential was recorded as experiments progressed. The active redox potentials and experimental endpoints were determined based on current and visual color changes.

Due to solution evaporation over time, the ratio of absorbance at 320 nm divided by the absorbance at 270 nm was determined in order to account for overall absorption increases. This value was then tracked throughout the experiments and proved to be a more stable indicator of reaction progress than absorbance alone. In addition, the visual change in solution color from clear to yellow and pink was also monitored as an indicator of degree of oxidation.

Stirred EGCG solutions in 0.1 M phosphate buffer with 0.1 M KCl at pH 6.0 were monitored. The open circuit potential was initially 220 mV. A. potential of 800 mV was applied, resulting in approximately 1 microamp of current in the cell. A duplicate solution was left uncovered on the table next to the electrochemical cell as the control. After seven hours, no change in either solution at 270 and 320 nm was found. At this point the experimental potential was increased to 1000 mV for a further 16 hours (Table 2A).

TABLE 2
Electrochemical manipulation of EGCG.

| | | | | |
|---|---|---|---|---|
| Sample ID | Time | 270 nm | 320 nm | 320 nm/270 nm |

A

| | | | | |
|---|---|---|---|---|
| Experiment | 23 hours | 0.418 | 0.107 | 0.256 |
| Control | 23 hours | 0.424 | 0.072 | 0.170 |

B

| | | | | |
|---|---|---|---|---|
| Control | 43 hours | 0.448 | 0.078 | 0.174 |
| Experiment | 43 hours | 0.432 | 0.171 | 0.396 |
| Control | 55 hours | 0.471 | 0.084 | 0.178 |
| Experiment | 55 hours | 0.422 | 0.197 | 0.467 |
| Control | 68 hours | 0.438 | 0.083 | 0.189 |
| Experiment | 68 hours | 0.429 | 0.224 | 0.522 |

C

| | | | | |
|---|---|---|---|---|
| Experiment | 73 hours | 0.430 | 0.229 | 0.533 |
| Experiment | 79 hours | 0.426 | 0.229 | 0.538 |
| Experiment | 120 hours | 0.436 | 0.238 | 0.546 |

These data demonstrate the increase in 320 nm quinone absorbance and associated decrease in the 270 nm phenol absorbance associated with oxidation of the EGCG over 16 hours at 1000 mV. The control sample did not oxidize, which is consistent with the known stability of EGCG under acidic conditions. After the combined time of 23 hours (7 h plus 16 h), the potential of the cell was increased to 1500 mV (Table 2B).

At 68 hours, the control sample was still clear, but the experimental solution had a feint yellowish color. The pH of the solutions remained 6.0 throughout the experiment. All data support the forced oxidation of EGCG at pH 6.0. Solutions were allowed to sit for two more days to observe the stability of the electrochemically-induced changes (Table 2C).

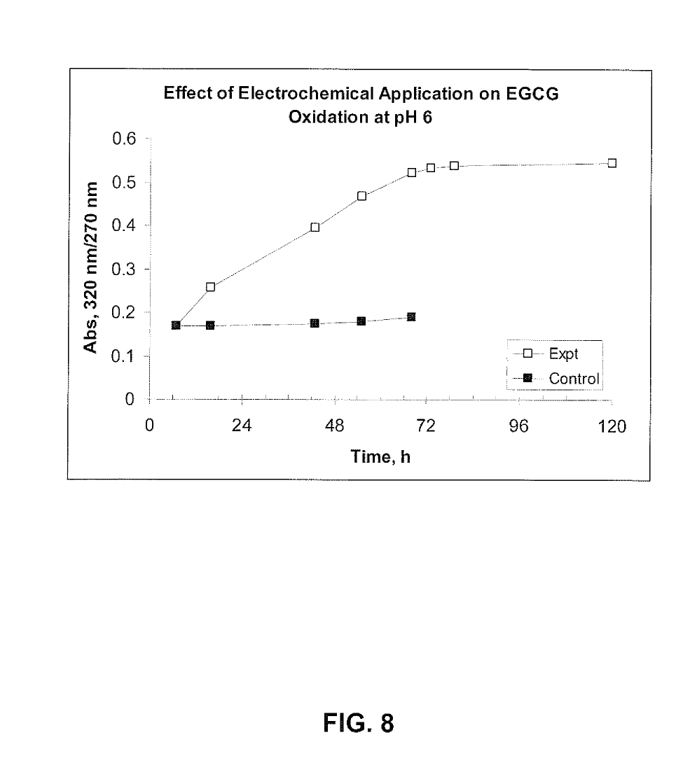

These data indicate that the solutions are stable for over two days without a significant increase or decrease in 320 nm absorbance over time. A summary of these data is presented in FIG. 8, where the effect of applied electrical potentials EGCG solutions at pH 6 is exhibited. After 7 h of 800 mV potential when no changes were detected, the potential was increased to 1000 mV until 23 h had elapsed, and further increased to 1500 mV until 68 h had elapsed. After 68 h, the solutions remained stirring with no current until 120 h had elapsed.

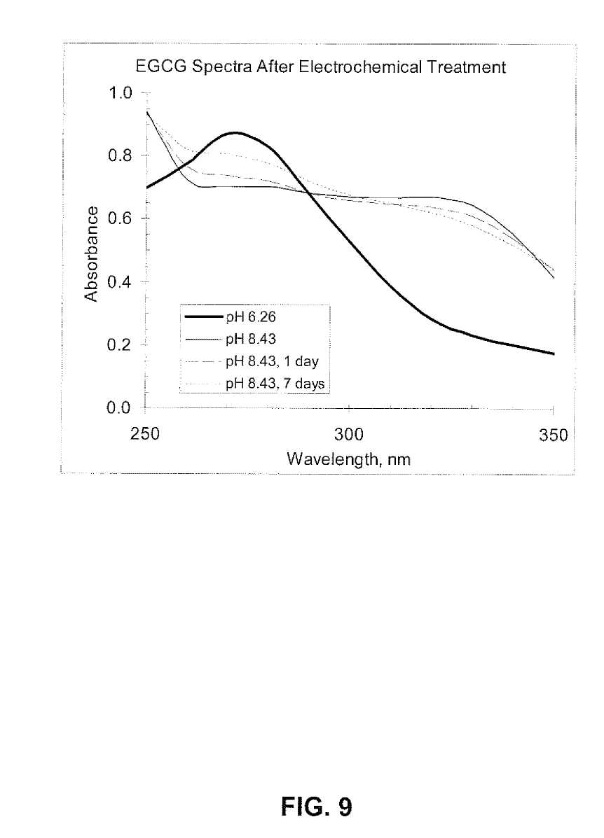

Solutions were pH modified following electrochemical oxidation to observe stability. The spectra of samples prepared from an EGCG solution at pH 6.26 under 1500 mV potential for 11.4 hours are shown in FIG. 9. The pH 6.26 solutions could not be compared to control samples because control samples at pH 6.26 did not oxidize. The figure shows the samples were stable after one day, and in the case of pH 8.43, it was relatively stable after 7 days. These samples were open to air, and no other precautions were taken to prevent oxidation by atmospheric oxygen.

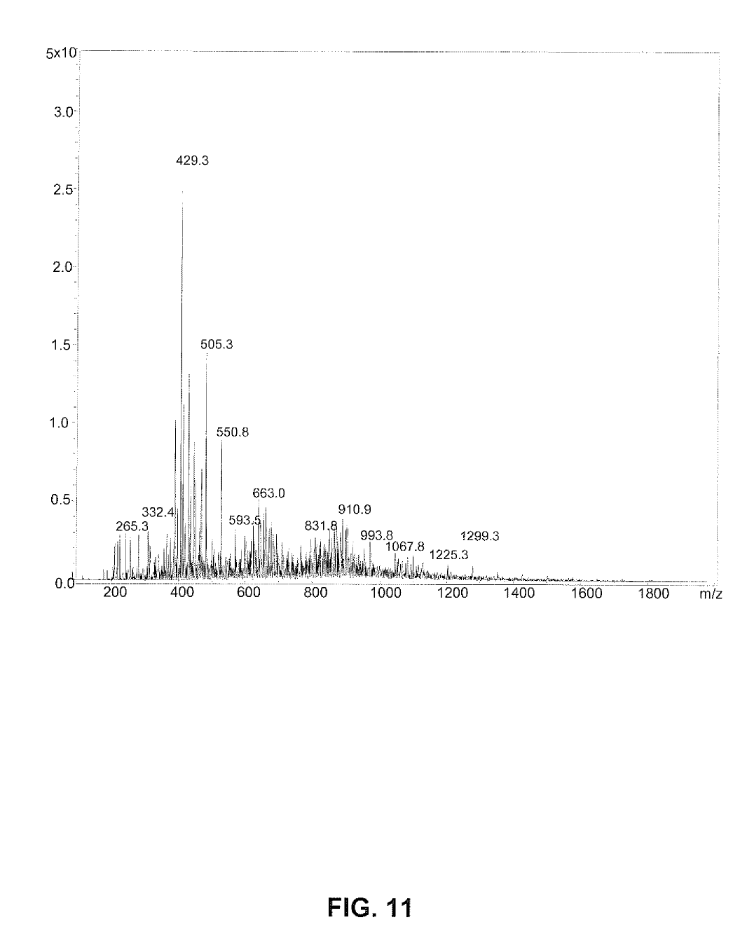

In FIG. 11, the compound with mass 911 has been identified as the EGCG dimer quinone, a predictable oxidation product of EGCG. These masses, and mass 911 in particular, are also present in the electrochemically oxidized sample analyzed by direct infusion mass spectrometry, indicating the formation of analogous larger molecules by phenolic coupling and free radical addition mechanisms.

### EXAMPLE VI

### Rationale and Application of the Concept

Experiments were performed attempting to show efficacy of oxidation on a given compound and its effect in vitro against herpes simplex viruses. Herpes simplex viruses were titrated by inoculation of 10-fold dilutions (HSV-1 was inoculated into Vero cell cultures, and HSV-2 was inoculated into CV1 cultures) in 96-well microliter tissue culture plates (Becton Dickinson Labware, Oxnard, Calif.). A vims dilution (0.1 ml) in RPMI 1640 with 1% fetal bovine serum (MM) was inoculated into each well with three wells per dilution. The plates were kept for 2 to 5 days, depending on the virus, and examined daily for cytopathic effect. Virus titers were calculated by the method of Reed and Muench (Am J Hyg 27:493-497, 1938).

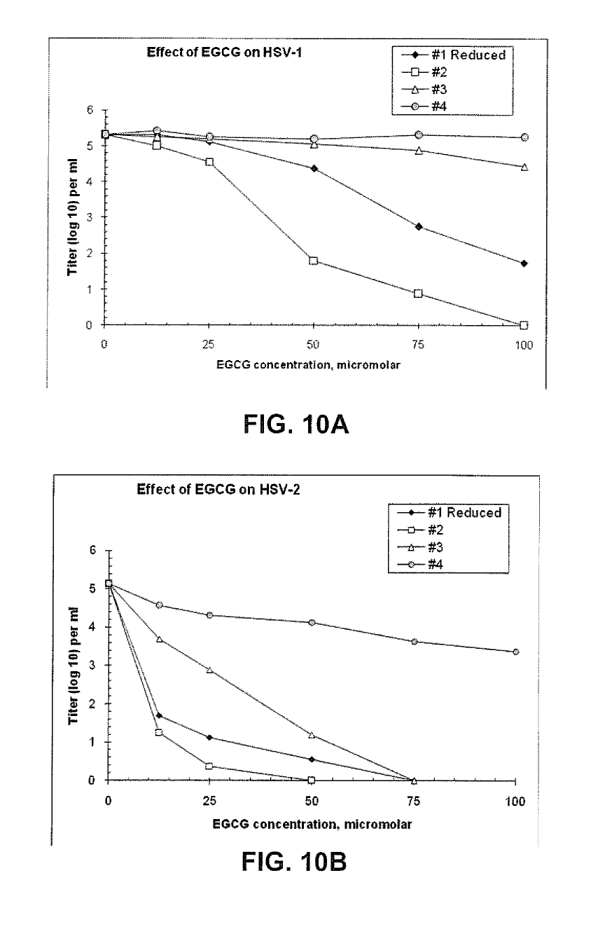

Assay of antiviral activity: About 10⁵ 50% tissue culture infective doses (TCID₅₀s) of virus were mixed with varying concentrations (12.5, 25, 50, 75 and 100 μM) of reduced and oxidized EGCG in MM and incubated at 37° C. for 30 min. The following samples were tested: The reduced form of EGCG (#1); partially oxidized form of EGCG (#2); the quinone or fully oxidized form of EGCG with the addition of a copper catalyst (#3); and quinone or fully oxidized form of EGCG with the addition of a zinc catalyst (#4). In Samples #3 and #4, the addition of the copper and zinc catalysts, respectively, raised the pH to 9.0-9.5. These compounds were totally oxidized, thereby taking a quinone form. These samples polymerized, turned a dark brown color with a heavy amount of precipitate. Once the reaction started there was no way to stop it. One basis for the present invention is to stop and control reactions in the partially oxidized semiquinone form, without progressing all the way through to the quinone form. The pH of the solution was 4.0-4.5. Sample #2 was a light orange color, evidencing oxidation and oligomerization. Virus mixed with MM alone was used as a control. After incubation, the infectivity of each mixture was titrated by the serial dilution endpoint method. Dilutions (10-fold) were made in MM. The 10⁻¹ to 10⁻⁵ dilutions were inoculated into monolayers of Vero or CV1 cells, and the vims titers were determined as described above. The difference between the titer (log 10) of the control vims and the titers of EGCG-virus mixtures, i.e. the reduction of virus titer, was used as a measure of antiviral activity. The oxidized form of EGCG showed the highest efficacy (FIG. 10).

As used in this specification and in the appended claims, the singular forms include the plural forms. For example the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and genetically herein. Each of the narrower species and sub-generic groupings falling within the scope of the generic disclosure also form part of these inventions. This includes the generic description of each invention with a proviso or negative limitation, removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

### REFERENCES

Hamza, A., and C-G Zhan. 2006. How can (−)-epigallocatechin gallate from green tea prevent HIV-1 infection Mechanistic insights from computational modeling and the implication for rational design of Aanti-HIV-1 entry inhibitors. J. Phys. Chem. B 110: 2910-2917.
Kauffmann et al., 2002. Influence of redox-active compounds and PXR-activators on human MRP1 and MRP2 gene expression. Toxicology 171(2): 137-146.
Kilmartin, P. A., and C. F, Hsu, 2003. Characterisation of polyphenols in green, oolong, and black teas, and in coffee, using cyclic voltammetry. Food Chem. 82: 501-512.
Oliveira et al., 1999. Electrochemical oxidation of mitoxantrone at a glassy carbon electrode. Analytica Chimica Acta 385(1): 401-408.
Ozkan et al., 2002. Electrochemical reduction and oxidation of the antibiotic cefepime at a carbon electrode. Analytica Chimica Acta 457(2); 265-274.
   Reed, L. J., and M. Muench. 1938. A simple method of estimating 50 percent end points. Am J Hyg. 27: 493-497.
Savaraj et al., 2005. Redox regulation of matrix metalloproteinase gene family in small cell Lung cancer cells. Free Radical Research 39(4): 373-381.

We claim:

1. A container for maintaining a particular oxidation state of a compound, the container comprising an anode and a cathode in electrically conductive contact with the compound, the anode and cathode being in electrical contact with a source of electromotive force, the source supplying sufficient electromotive force to maintain the compound in a preferred or desired redox state, when in contact with the anode or cathode.

2. The container of claim 1 wherein the source of electromotive force is a battery, piezoelectric or other voltage source.

3. The container of claim 1 wherein the anode has greater surface area than the cathode.

4. The container of claim 1 wherein the cathode has greater surface area than the anode.

5. The container of claim 1 wherein the container further comprises a redox electron sink.

6. The compound of claim 1, wherein the compound may be utilized as an antiviral, anticancer, antibacterial or antimicrobial agent.

7. An applicator for preparing an oxidizable or reducible compound in a desired redox state comprising:
a) dissolving or suspending the compound in an electrically conductive solution;
b) contacting the solution containing the compound with an anode and a cathode; and
c) supplying an electromotive force to the anode and cathode, said force being sufficient to oxidize or reduce the compound to a preferred or desired state, whereby such preferred or desired state of the compound is prepared and administered.

8. The applicator of claim 7 is one selected from the group consisting of:
a) a skin patch;
b) an eyedropper; and
c) a syringe
for control of redox state during application in a process called electrodynamic therapy.

9. A method of treating a patient suffering from a virus, the method comprising the steps of;
(1) providing a compound in a reduced form;
(2) altering the compound's oxidation state to a more oxidized form;
(3) maintaining a partially oxidized form of the compound; and
(4) administering the partially oxidized form of the compound to the patient suffering from the virus.

10. The method of claim 9, wherein the compound is epigallocatechin gallate.

11. The method of claim 9, wherein the partially oxidized form of the compound is the semiquinone form of epigallocatechin gallate.

12. The method of claim 9, wherein the virus is herpes simplex vims.

13. The method of claim 9, wherein the virus is AIDS.

14. The method of claim 9, wherein the patient is a mammal.
